(19) United States
(12) Patent Application Publication
(10) Pub. No.: US 2023/0245740 A1
(43) Pub. Date: Aug. 3, 2023

(54) WEB-BASED PHARMACOGENOMICS TOOL

(71) Applicant: DASMAN DIABETES INSTITUTE, DASMAN (KW)

(72) Inventors: FAHD AL-MULLA, DASMAN (KW); SUMI ELSA JOHN, DASMAN (KW); ARSHAD MOHAMED CHANNANATH, DASMAN (KW); PRASHANTHA HEBBAR, DASMAN (KW); RASHEEBA IQBAL, DASMAN (KW); ALPHONSE THANGAVEL, DASMAN (KW)

(21) Appl. No.: 17/587,195

(22) Filed: Jan. 28, 2022

(51) Int. Cl. G16H 20/10; G16H 10/40; G16H 10/60; G16H 50/70; G16H 15/00

(52) U.S. Cl. CPC G16H 20/10; G16H 10/40; G16H 10/60; G16H 50/70; G16H 15/00

(57) ABSTRACT

A system and method of providing personalized medication dosing recommendations are provided. The web-based pharmacogenomics tool includes a system and method for analyzing a patient's genomic information and producing a personalized report containing recommendations to adjust medication selection and dosing for the patient based upon known pharmacogenomic interactions. The tool is adapted to receive a whole genome screening file from a user, use Stargazer to detect diplotypes in specific pharmacogenes: assigning allele functionality, phenotype, drug names, and recommended dosage information based upon the detected diplotypes, and generating a personalized pharmacogenomics report including recommendations of medications to use or avoid and/or suggested dosing adjustments. The tool allows users to submit genomic information in either hg48 or hg19 format.

### BACKGROUND

### 1. Field

The disclosure of the present patent application relates to a system and method of analyzing genetic information to provide personalized medication dosing recommendations, and particularly to a web-based pharmacogenomics tool for analyzing a patient's genetic information and producing a personalized report containing recommendations to adjust medication selection and dosing based upon known pharmacogenomic interactions.

### 2. Description of the Related Art

Pharmacogenomics plays an instrumental role in drug safety and efficacy. Studies indicate that the most commonly prescribed pharmaceuticals are effective in only 25% to 60% of patients. Furthermore, each year, hospitals in the United States report more than two million patients with adverse drug reactions (ADRs), resulting in up to 100,000 fatalities and a total cost of up to $5.6 million per hospital. In a multicenter study by Pirmohamed et al., ADRs were found to account for 6.5% of hospitalizations in two large hospitals in the United Kingdom. Interestingly, almost 100% of the population carries at least one actionable genetic variant. Haplotypes are groups of variants in a person's genome that are inherited together. Some of the conditions known to affect a person's response to certain drugs include warfarin resistance, warfarin sensitivity, clopidogrel resistance, malignant hyperthermia, Stevens-Johnson syndrome/toxic epidermal necrolysis and thiopurine S-methyltransferase deficiency.

Genetic variants together with environmental factors play an important role in an individual's response to drug treatment. As sequencing has become more affordable, many health centers can now easily get patient genomes sequenced. Genetic markers in pharmacogenomics are identified by means of numbers and letters and separated from gene names by a star known as star allele nomenclature. For example, CYP2B6*2 identifies the genetic variant in gene CYP2B6 at genomic position g.5071C>T, leading to amino acid substitution R22C. (Lana. T., et al., 2001) Star allele nomenclature has become the gold standard in pharmacogenomics as it helps standardize the identification of pharmacogenetic alleles better and helps to avoid transcription mistakes, which are more common when using Human Genome Variation Society (HGVS) nomenclature. In pharmacogenomics, accurate detection of star alleles in clinically actionable pharmacogenes provides the foundation for phenotype prediction and treatment decisions.

Custom-designed pharmacogenomic arrays were the technology of choice for their ability to provide faster, cost-effective solutions, particularly for large sample sizes as part of research studies. The Affymetrix-developed Drug Metabolizing Enzymes and Transporters (DMET) Plus array was one of the first pharmacogenomic arrays, which was implemented in two PGx initiatives, the 1200 patients Project and the PG4KDS protocol. Microarrays were successfully deployed in several other pre-emptive pharmacogenomics initiatives such as Pharmacogenomic Resource fir Enhanced Decisions in Care and Treatment (PREDICT) using Illumina's VeraCode ADME core panel (Illumina, Inc. San Diego, Calif. ESA) and in five U.S. medical centers.

However, there are several drawbacks to this technology, in the context of pharmacogenomics testing. Firstly, novel variants of potential clinical relevance are not taken into consideration while using pharmacogenomic arrays. Several studies have shown that rare variants comprise 30/40% of the variation in pharmacogenes. Secondly, due to the difference in test designs across different platforms, it becomes difficult to compare results, often leading to inconsistent haplotype calling for the same alleles. Another problem that has been reported with the use of PGx arrays is in the identification of copy number variations (CNV).

Next-generation sequencing (NGS) approaches are gaining more popularity in PGx, involving either pharmacogene-targeted/whole exome sequencing (WES) (Price, M. J. et al. 2012) or whole genome sequencing (WGS). The advantage of WGS is that in a single assay, it can detect not only disease-causing hut also pharmacogenetically relevant variants. Patrinos et al., in their study analyzing 482 whole genome sequences, demonstrated the pre-eminence of WGS over other genetic screening methods to accurately, determine an individual's pharmacogenomic profile in a comprehensive manner. A distinctive benefit of NGS technology is the ability to detect novel and rare variants in the genome that might be missed in an array. Furthermore, it yields better quantitative results with somatic variation as compared with Sanger sequencing technology and result in a higher throughput scale. Whole exome sequencing, though it may appear as a viable choice compared to whole genome sequencing in terms of cost, fails to capture the regulatory and untranslated regions in the genome where many PGx variants reside. To further complicate choices, the efficiency of commercial target kits varies considerably, leaving a significant proportion of variants undetected. Several studies, including that by Reisberg et al., have concluded that whole exome sequencing is not suitable for pharmacogenomic predictions. (Reisberg et al., “Translating genotype data of 44,000 biobank participants into clinical pharmacogenetic recommendations: Challenges and solutions.” Genet. Med. 21: pp. 1345-1354 (2019))

One major challenge in the implementation of pharmacogenomics is the retrieval of genotypic marker information in star allele diplotype format. Some of the pharmacogenomic translation took that are currently in use include Astrolabe, Aldy, Stargazer (University of Washington) and PharmCAT. Except for PharmCAT, the other three tools work only in Linux and Mac Operating Systems and their output includes diplotypes, phenotypes, suballeles and novel Single-nucleotide variants (SNV). While Astrolabe allows both GRCh37 and GRCh38 input file formats. Aldy and Stargazer can only accept files in GRCh37 format, whereas PharmCAT allows Variant Call format (VCF) only in GRCh38 format. Also, among the tour, only PharmCAT provides drug guideline recommendations.

The next challenge is the translation of genetic test results into clinical action. PharmGKB® has published PGx-based drug dosing guidelines by several consortia, including the Clinical Pharmacogenetics Implementation Consortium (CPIC®), the Dutch Pharmacogenetics Working Croup (DPWG), the Canadian Pharmacogenomics Network for Drug Safety (CPNDS) and other professional societies that provide therapeutic recommendations for well-known pharmacogene-drug pairs. A comparison study between CPIC® and DPWG guidelines reported substantial similarities and few observed differences that could lead to the use of different methodologies for drug dosing.

Finally, the success of PGx implementation relies heavily on its acceptance among patients and clinical healthcare professionals. The major stumbling block to its widespread implementation among general physicians and clinical geneticists appears to be the lack of knowledge of genetics and an unfamiliarity with PGx data and tools. CDS delivered through electronic health records (EHRs) has proved indispensable in facilitating gene-based drug prescription for patient care.

Thus, a web-based pharmacogenomics tool solving the aforementioned problems is desired.

### SUMMARY

The presently described web-based pharmacogenomics tool relates to a system and method for analyzing a patient's genomic information and producing a personalized report containing recommendations to adjust one or more medication selection and dosing instructions for the patient based upon known pharmacogenomic interactions. The tool is adapted to receive a whole genome screening file from a user: use Stargazer to detect diplotypes in specific pharmacogenes: assign allele functionality, phenotype, drug names, and recommended dosage information based upon the detected diplotypes and a set of stored pharmacogenomic data: and generate a personalized pharmacogenomics report personalized for the patient including recommendations of medications to use or avoid and/or suggested dosing adjustments to one or more medications. The tool allows users to submit genomic information in either hg38 or hg19 format.

In accordance with one aspect of this disclosure, a system for providing a web-based pharmacogenomics tool includes a server for hosting a website accessible through the Internet: said website adapted to receive information from a remote user, said information transmitted through the internet and including a whole genome sequence (WGS) file for a patient and said server adapted to communicate information to the user in the form of a personalized pharmacogenomics report: non-transitory memory configured to store executable instructions and pharmacogenomic data: and a processor in communication with the server and the non-transitory memory: Wherein the pharmacogenomic data includes allele functionality, phenotype, drug names, and recommended dosage information, for example as retrieved from PharmGKB® and CPIC® resources: and wherein the processor is adapted to receive the WGS sequence file, use Stargazer to extract diplotypes in specific pharmacogenes from the WGS sequence file, assign allele functionalities, phenotype, drug names, and recommended dosage information based upon the extracted diplotypes and the stored pharmacogenomic data, generate a personalized pharmacogenomics report for the patient including recommendations of medications to use or avoid and/or suggested dosing adjustments to at least one medication, and deliver the report to the user.

In accordance with a further aspect of this disclosure, a method for providing a web-based pharmacogenomics tool includes providing a website accessible through the Internet, said website adapted to receive information from a user including a whole genome sequence (WGS) file for a patient; and upon receiving a WGS sequence file, using Stargazer to extract diplotypes in specific pharmacogenes from the WGS sequence file: assigning allele functionality, phenotype, drug names, and recommended dosage information based upon the extracted diplotypes and stored pharmacogenomic information: generating a personalized pharmacogenomics report including recommendations of medications for the patient to use or avoid and/or suggested dosing adjustments to at least one medication: and delivering the report to the user.

These and other features of the present subject matter will become readily apparent upon further review of the following specification.

### BRIEF DESCRIPTION OF THE DRAWINGS

FIG. 1 depicts a pie chart of the distribution of 37 pharmacogenomic drugs by class.

FIG. 2A depicts a flowchart of the PharmaKU software process, including receiving a single sample WGS VCF file from a user: using Stargazer to detect diplotypes in the nine pharmacogenes: and assigning the corresponding allele functionality, phenotype, drug names, and recommended dosage information, followed by generating a personalized pharmacogenomics report.

FIG. 2B depicts the first section of the personalized pharmacogenomics report: including genes of interest, genotypes, allele functionality, phenotypes, and clinical recommendations.

FIG. 2C depicts the second section of the personalized pharmacogenomics report, containing a detailed discussion of the genotype results reported in the first section.

FIG. 3 depicts a graph of metabolizer topes identified for seven major pharmacogenes from 20 individual reports.

Similar reference characters denote corresponding features consistently throughout the attached drawings.

### DETAILED DESCRIPTION OF THE PREFERRED EMBODIMENTS

### Definitions

As used herein “Pharmacogenomics” (PGx) is the field that studies how genetic makeup affects a person's response to drugs. Even though the concept of pharmacogenomics has been around since the 1950s, it is only now that we witness its proper integration with clinical informatics for clinical decision support (CDS). Advancements in array-based and high-throughput sequencing technologies have enabled scientists to quickly profile an individual's genetic make-up, which can be used to query pharmacogenomics resources.

As used herein. “genetic variant” refers to a substitution of one or more nucleotides at a specific position in the genome. Genetic variants may be single-nucleotide variants (a variation at a single nucleotide position) or multiple-nucleotide variants (any variation including more than one variable nucleotide position). A genetic variant gives rise to at least two alleles, with each allele referring to one of the two or more version of the same gene at the particular position in the genome that has mutated to form the genetic variant.

As used herein, a “haplotype” is a group of alleles that are inherited together. Thus, in organisms having paired chromosomes, the haplotype refers to the chromosomes inherited on a single set of chromosomes, while the term “diplotype” refers to both sets of chromosomes (and thus to all alleles present on the chromosome pairs for a particular individual). Thus, a diplotype may be thought of as a specific combination of two haplotypes. Notably, in pharmacogenomics, a haplotype is commonly used to refer to a combination of alleles found in a single gene: while in other contexts a haplotype may refer to alleles inherited together and located in different genes.

As used herein “hg 38 format” or “GRCh38” refers to Genome Reference Consortium Human Build 38, a full reference genome for Homo sapiens sequenced by the Human Genome Project.

As used herein “hg 19 format” or “GRCh37” refers to Genome Reference Consortium Human Build 37, an alternative full reference genome for Homo sapiens sequenced by the Human Genome Project.

As used herein a “drug” or “medication” refers to either prescription drugs or over the counter medications for which a dosing schedule has been or will be recommended by a physician or health care provider.

As used herein. “dosing adjustments” or “suggested dosing adjustments” include not just adjusting the amount or dosing of a medication administered to the patient hut can also include the selection and/or deselection of specific medication(s) to be administered or previously administered to the patient.

### PharmaKU

The Web-based pharmacogenomics tool described herein relates to a system and method for analyzing a patient's genomic information and producing a personalized report containing, recommendations for the patient to adjust one or more medication selection and dosing instructions based upon known pharmacogenomic interactions. The tool is adapted to receive a whole genome screening file from a user: use Stargazer to detect diplotypes in specific pharmacogenes: assign allele functionality, phenotype, drug names, and recommended dosage information based upon the detected diplotypes and a set of stored pharmacogenomic data: generate a personalized pharmacogenomics report including recommendations of medications for the patient to avoid author suggested dosing adjustments to one or more medications: and deliver the personalized pharmacogenomics report to the user. The tool allows users to submit genomic information in either hg38 or hg19 format. The user can be any of a licensed medical professional, a technician well versed in the PharmaKU system, and the patient who is or will be taking the recommended medication(s) in the recommended dosage(s). In some embodiments, the user and the patient will be the same person. In other embodiments, the user and the patient will be different people. In these latter embodiments, the user and the patient can either be in the same location or remote from one another.

In accordance with one aspect of this disclosure, a system for providing a web-based pharmacogenomics tool includes a server for hosting a website accessible through the Internet: said website adapted to receive information from a remote user, said information transmitted through the internet and including a whole genome sequence (WGS) file for a patient and said server adapted to communicate information to the user in the form of a personalized pharmacogenomics report for the patient: non-transitory memory configured to store executable instructions and pharmacogenomic data: and a processor in communication with the server and the non-transitory memory: wherein the pharmacogenomic data includes allele functionality, phenotype, drug names, and recommended dosage information, for example, as retrieved from PharmGKB® and CPIC® resources: and wherein the processor is adapted to receive the WGS sequence file, use Stargazer to extract diplotypes in specific pharmacogenes from the WGS sequence file, assign allele functionality, phenotype, drug names, and recommended dosage information based upon the extracted diplotypes and the stored pharmacogenomic data, generate a personalized pharmacogenomics report for the patient including recommendations of medications to use or avoid and/or suggested dosing adjustments to at least one medication, and deliver the report to the user.

In accordance with a further aspect of this disclosure, a method for providing a web-based pharmacogenomics tool includes providing a website accessible through the Internet, said website adapted to receive information from a user including a whole genome sequence (WGS) file for a patient: and upon receiving a WGS sequence file, using Stargazer to extract diplotypes in specific pharmacogenes from the WGS sequence file, assigning allele functionality, phenotype, drug names, and recommended dosage information based upon the extracted diplotypes and stored pharmacogenomic information, generating a personalized pharmacogenomics report for the patient including recommendations of medications to use or avoid and/or suggested dosing adjustments to at least one medication, and delivering the report to the user.

In an embodiment, the systems and methods disclosed herein may include extracting diplotypes from specific pharmacogenes. In a further embodiment, the specific pharmacogenes may be selected from the group consisting of CYP2B6, CYP2C19, CYP2C9, CYP2D6, CYP3A5, DPYD, SLCO1B1, TPMT, UGT1A1, and a combination thereof. In a further embodiment, the methods disclosed herein may include extracting diplotypes from CYP2B6, CYP2C19, CYP2C9, CYP2D6, CYP3A5, DPYD, SLCO1B1, TPMT, and UGT1A1.

In an embodiment, the systems and methods disclosed herein may include recommended dosing adjustments to at least one medication selected from the group consisting of amitriptyline, atazanavir, atomoxetine, capecitabine, celecoxib, citalopram, clomipramine, clopidogrel, codeine, desipramine, doxepin, efavirenz, escitalopram, fluorouracil, flurbiprofen, fluvoxamine, fosphenytoin, imipramine, lansoprazole, lornoxicam, meloxicam, nortriptyline, omeprazole, ondansetron, pantoprazole, paroxetine, phenytoin, piroxicam, sertraline, simvastatin, tacrolimus, tamoxifen, tenoxicam, trimipramine, tropisetron, voriconazole, warfarin, and any combination thereof.

In an embodiment, the system includes a computing system comprising non-transitory memory configured to store executable instructions and a processor (including hut not limited to a hardware processor or a virtual processor) in communication with the non-transitory memory, the processor programmed by the executable instructions to perform any of the methods disclosed herein.

In an embodiment, the methods disclosed herein may be stored as executable instructions on a computer readable medium; wherein said instructions when executed by a processor cause the processor to perform the method.

With the tremendous advancements in genome sequencing technology in the field of pharmacogenomics, there is a need for data to be made accessible to be more efficiently utilized by broader clinical disciplines. Physicians who require the drug-genome interactome information have been challenged by the complicated pharmacogenomic star-based classification system. The present system provides an end-to-end web-based pharmacogenomics tool, “PharmaKU”, which has a comprehensive easy-to-use interface. PharmaKU can help to overcome several hurdles posed by previous pharmacogenomics tools, including input in hg38 format only, while hg19/GRCh37 is now the most popular reference genome assembly among clinicians and geneticists, as well as the lack of clinical recommendations and other pertinent dosage-related information. This tool extracts genetic variants from nine well-annotated pharmacogenes (for which diplotype to phenotype information is available) from whole genome variant files and uses, for example. Stargazer software to assign diplotypes and apply prescribing recommendations from pharmacogenomic resources. The tool is wrapped with a user-friendly web interface, which allows for choosing hg19 or hg38 as the reference genome version and reports results as a comprehensive PDF document, or in another suitable format. PharmaKU is anticipated to enable bench to bedside implementation of pharmacogenomics knowledge by bringing precision medicine closer to a clinical reality.

Pharmacogenomics is a good example of integration of Precision Medicine in medical practice. By way of profiling an individual's genetic make-up through array-based and high throughput sequencing technologies, it is now possible to predict if a specific medicine will be effective in a person or likely to cause adverse drug reactions (ADRs).

Considering the current state of clinical pharmacogenomics together with the availability of pharmacogenomic resources, the present disclosure relates to a web-based tool that facilitates the easy transition of a person's whole genuine variant data into clinical recommendations. Through this, clinicians and geneticists can implement pharmacogenomics more broadly in patient care. The initial version of this software covers nine well annotated pharmacogenes that coyer the activity of 37 drugs.

Advances in NGS have revolutionized the field of pharmacogenomics by pinpointing genetic variants relevant to drug action and metabolism. It is rightly said that pharmacogenomics is a forerunner in bringing precision medicine to the clinic. However, identification of variants is only the first step toward better treatment. The availability of quality-controlled and patient-centered software to link the identified pharmacogenomic variants from an individual's genome to the existing knowledge of drug dosing guidelines holds the key to widespread and successful implementation of pharmacogenomics in our healthcare system. In a study that evaluated the impact of preemptive pharmacogenomic genotyping results, an institutional CDS system provided pharmacogenomic results using traffic light alerts. As a result, medications with high pharmacogenomic risk were changed and no high-risk drugs were prescribed during the entire study.

The presently described system can include any or all of a number of measures to minimize false-positive results using the herein described software. First, an important step in the pharmacogenomic translation process, prone to erroneous results, is the assigning of diplotypes. Three currently available public tools were shortlisted and benchmarked using in-house data. Based on the results (Table 2). Stargazer was incorporated into the present software for diplotype calling. Second, the remaining processes in the software pipeline deal with the mapping of assigned diplotype to a gene's allele functionality, phenotype, and dosage recommendation. This information was retrieved directly from PharmGKB® and CPIC® resources, without using any third-party software, thereby minimizing chances of data corruption. Third, any prescription recommendations made must follow a standard, wherein only drugs having CPIC® guidelines and with pre- and post-test alert flowcharts in PharmGKB® are included in the software. This was done to minimize discrepancies in naming and dosage information across different guidelines.

As of February 2021, dosage recommendations for nine pharmacogenes and 37 drugs have been incorporated into the presently described software, the number of drugs for which prescription information is available is limited by the information provided by the CPIC® guidelines. This software can be updated with more genes in a timely manner. The drug dosing guidelines published by DPWG and CPNDS may also be adopted and these recommendations made available through future versions of the presently described software. No other web-based, publicly available pharmacogenomics software allows for VCF inputs in both hg19 and GRCh38 format. Through this effort, the pharmacogenomic translation process has been simplified to the advantage of physicians, that with a single click, they are provided with a comprehensive pharmacogenomic report of their patient, complete with prescribing recommendations.

More than one third of the drugs for which PGx dosing recommendations are available belong to the class of antidepressants. Lack of pharmacogenomic data on other commonly used drug classes, such as alimentary tract and metaholisnt-related drugs (13%), cancer drugs (11%) and cardiovascular/lipid-modifying drugs (3%), will shed more light on the necessity for more studies in this field.

Although the cost of WGS continues to decline, it remains prohibitively expensive for widespread clinical use. However, its use is justified by the fact that a one-time genomic test to determine a person's pharmacogenomic profile would inform clinicians about dosing and effectiveness for a multitude of drugs. Inclusion of this information into the EHR would be invaluable to patients throughout their lifetime.

The following examples illustrate the present teachings

### Example 1

### Developing PharmakU

Gene-specific information tables provided jointly by PharmGKB® and CPIC® were used to finalize the pharmacogenes used in this tool. We restricted the number of genes to only those for which a diplotype to phenotype information table was available and to those which were common to the 28 genes mentioned in the study by Lee et al. describing the utility of Stargazer on whole genome sequences.

Genetic markers in pharmacogenomics are indicated using star-allele nomenclature-numbers and letters and separated from the gene name by a star. Several bioinformatics software tools that aid in the conversion of a genome variants to star-allele nomenclature are available including Astrolabe, PharmCAT and Stargazer. We examined concordance in calling star-allele nomenclature, by way of testing these tools on 20 in-house hole-genomes with coverage greater than 30× to select the tool most suitable for our purpose.

We used the diplotype-phenotype table, from the gene-specific information tables, to map the sample diplotype assigned in the previous step to its phenotype. Allele functionality data was also obtained from the diplotype-phenotype table. Medication/drug name information for the corresponding gene was obtained from the Clinical Pharmacogenetics Implementation Consortium (CPIC®) of the National Institutes of Health's Pharmacogenomics Research Network (http://www.pgrn.org) (accessed on 7 Sep. 2020). Drug dosage information was retrieved from the Pharmacogenomics Knowledge Base (PharmGKB®, http://www.pharmgkb.org) (accessed on 15 Dec. 2020).

PharmaKU was implemented in Python3 and uses a Django web framework. It was deployed in Apache and mod_wsgi. PharmaKU is supported by all major browsers.

We chose 9 out of the 18 pharmacogenes listed in the gene-specific information tables based on the standard annotations that are available for each gene (Table 1).

TABLE 1
List of Nine Pharmacogenes used in PharmaKU Along with
Associated Drugs

| | | | CPIC ® Publica- |
|---|---|---|---|
| Gene | Drug | PGx on FDA Label | tions (PMID) |
| CYP2B6 | efavirenz | Actionable PGx | 31006110 |
| CYP2C19 | amitriptyline | | 23486447; |
| | | | 27997040 |
| | citalopram | Actionable PGx | 25974703 |
| | clopidogrel | Actionable PGx | 21716271; |
| | | | 23698643 |
| | escitalopram | Actionable PGx | 25974703 |
| | lansoprazole | Informative PGx | 32770672 |
| | omeprazole | Actionable PGx | 32770672 |
| | pantoprazole | Actionable PGx | 32770672 |
| | voriconazole | Actionable PGx | 27981572 |
| | clomipramine | | 23486447; |
| | | | 27997040 |
| | dexlansoprazole | Actionable PGx | 32770672 |
| | doxepin | Actionable PGx | 23486447; |
| | | | 27997040 |
| | imipramine | | 23486447; |
| | | | 27997040 |
| | sertraline | | 25974703 |
| | trimipramine | | 23486447; |
| | | | 27997040 |
| | esomeprazole | Actionable PGx | 32770672 |
| | rabeprazole | Actionable PGx | 32770672 |
| CYP2C9 | celecoxib | Actionable PGx | 32189324 |
| | flurbiprofen | Actionable PGx | 32189324 |
| | fosphenytoin | | 25099164; |
| | | | 32779747 |
| | ibuprofen | | 32189324 |
| | lornoxicam | | 32189324 |
| | meloxicam | Actionable PGx | 32189324 |
| | phenytoin | Actionable PGx | 25099164; |
| | | | 32779747 |
| | piroxicam | Actionable PGx | 32189324 |
| | tenoxicam | | 32189324 |
| | warfarin | Actionable PGx | 21900891; |
| | | | 28198005 |
| | aceclofenac | | 32189324 |
| | aspirin | | 32189324 |
| | diclofenac | | 32189324 |
| | indomethacin | | 32189324 |
| | lumiracoxib | | 32189324 |
| | nabumetone | | 32189324 |
| | naproxen | | 32189324 |
| CYP2D6 | amitriptyline | Actionable PGx | 23486447; |
| | | | 27997040 |
| | atomoxetine | Actionable PGx | 30801677 |
| | codeine | Actionable PGx | 22205192; |
| | | | 24458010 |
| | nortriptyline | Actionable PGx | 23486447; |
| | | | 27997040 |
| | ondansetron | Informative PGx | 28002639 |
| | paroxetine | Informative PGx | 25974703 |
| | tamoxifen | Actionable PGx | 29385237 |
| | tropisetron | | 28002639 |
| | clomipramine | Actionable PGx | 23486447; |
| | | | 27997040 |
| | desipramine | Actionable PGx | 23486447; |
| | | | 27997040 |
| | doxepin | Actionable PGx | 23486447; |
| | | | 27997040 |
| | fluvoxamine | Actionable PGx | 25974703 |
| | imipramine | Actionable PGx | 23486447; |
| | | | 27997040 |
| | trimipramine | Actionable PGx | 23486447; |
| | | | 27997040 |
| CYP3A5 | tacrolimus | | 25801146 |
| DPYD | capecitabine | Actionable PGx | 23988873; |
| | | | 29152729 |
| | fluorouracil | Actionable PGx | 23988873; |
| | | | 29152729 |
| | tegafur | | 23988873; |
| | | | 29152729 |
| SLCO1B1 | simvastatin | | 22617227; |
| | | | 24918167 |
| TPMT | azathioprine | Testing recommended | 21270794; |
| | | | 23422873; |
| | | | 30447069 |
| | mercaptopurine | Testing recommended | 21270794; |
| | | | 23422873; |
| | | | 30447069 |
| | thioguanine | Testing recommended | 21270794; |
| | | | 23422873; |
| | | | 30447069 |
| UGT1A1 | atazanavir | | 26417955 |

We compared three pharmacogenomic translation tools for calling diplotypes across the nine pharmacogenes in Table 1 in 20 WGS samples. We found that Stargazer called diplotypes in more genes: 92.2% of the cases compared with Astrolabe (33.3%) and PharmCAT (31.1%) (Table 2). We also observed better concordance in results between Stargazer and Astrolabe and Stargazer and PharmCAT than between Astrolabe and PharmCAT in any single sample. For these reasons, we decided to implement Stargazer version 1.2.2 in our software for calling star alleles from the nine pharmacogenes using WGS data. We have also assessed five samples independently using two different technologies: Illumina's pharmacogenetic-targeted panel and whole genome sequence data. Scoring showed 100% accuracy between the two methods (data not shown).

TABLE 2
Comparison of Diplotype Detected in Nine Pharmacogenes Using Astrolabe.
PharmCAT and Stargazer in 20 Whole Genome Sequencing (WGS) Samples.

| | | | | | | Gene | | | | |
|---|---|---|---|---|---|---|---|---|---|---|
| ID | Tool | CYP2B6 | CYP2C9 | CYP2C19 | CYP2D6 | CYP3A5 | DPYD | SLC01B1 | TPM1 | UGT1A1 |
| 1 | Astrolabe | | *1/*2 | *1/*1 | *2/*4 | | | | | |
| | PharmCAT | | *1/*2, *1/*35 | | | | | *5/*20, *5/*21 *1/*5 | | *36, *60, *60 |
| | Stargazer | *1/*2 | *1/*2 | *1/*1 | *2/*4 | *3/*3 | *S12/*S12 | | *1/*1 | |
| 2 | Astrolabe | | *1/*1 | *1/*17 | *2/*41 | | | | | |
| | PharmCAT | | | *1/*4B, *1/*17 | | | | *1A/*18 | | *60/*60 |
| | Stargazer | *1/*1 | *1/*1 | *1/*17 | *2/*119 | *3/*3 | *6/*S12 | *1/*1B | *1/*1 | *60/*60 |
| 3 | Astrolabe | | *1/*1 | *2/*2 | *41/*86 | | | | | |
| | PharmCAT | *2/*2 | | | | | | *19/*20, *19/*21 | | *36, *60 |
| | Stargazer | *1/*6 | *1/*1 | *2/*2 | *86/*119 | *3/*3 | *1/*9A | *1/*1B | *1/*1 | |
| 4 | Astrolabe | | *1/*1 | *1/*17 | *1/*41 | | | | | |
| | PharmCAT | | | *1/*4B, *1/*17 | | | | | | *36, *60 |
| | Stargazer | *1/*1 | *1/*1 | *1/*17 | *1/*119 | *1/*3 | *S3/*5 | *1/*14 | *1/*1 | |
| 5 | Astrolabe | | *1/*1 | *1/*2 | *10/*4 | | | | | |
| | PharmCAT | | | *1/*2 | | | | *1A/*20, *1A/*21 | | *79/*79 |
| | Stargazer | *1/*22 | *1/*1 | *1/*2 | *4/*10 | *1/*3 | *S3/*S12 | *1/*1B | *1/*1 | |
| 6 | Astrolabe | | *1/*1 | *1/*2 | *1/*86 | | | | | |
| | PharmCAT | | | *1/*2 | | | | *1A/*18 | | *60/*60 |
| | Stargazer | *5/*6 | *1/*1 | *1/*2 | *1/*1 | *3/*3 | *S3/*S12 | *1/*1B | *1/*1 | |
| 7 | Astrolabe | | *2/*17 | *1/*1 | *1/*2 | | | | | |
| | PharmCAT | | | *2/*4B, *2/*17 | | | | Multiple | | |
| | Stargazer | *1/*1 | *1/*1 | *2/*17 | *1/*2 | *3/*3 | *1/*S12 | *1/*S461 | *1/*1 | *79/*79 |
| 8 | Astrolabe | | *1/*1 | *1/*1 | *1/*10 | | | | | |
| | PharmCAT | | | | | | | *18/*18, *18/*19, *19/*19 | | *60 |
| | Stargazer | *6/*6 | *1/*1 | *1/*1 | *1/*10 | *1/*3 | *S12/*S38 | *1/*1 | *1/*1 | *60/*79 |
| 9 | Astrolabe | | *1/*2 | *1/*1 | *1/*4 | | | | | |
| | PharmCAT | | | *1/*2 | | | | *1A/*18, *1A/*19 | | *36, *60 |
| | Stargazer | *1/*1 | *1/*1 | *1/*2 | *1/*4 | *3/*3 | *S12/*S12 | *1/*1 | *1/*1 | |
| 10 | Astrolabe | | *2/*2 | *1/*1 | *1/*4 | | | | | |
| | PharmCAT | | | *2/*2 | | | | *20/*20, *20/*21, *21/*21 | | *60/*60 |
| | Stargazer | *6/*6 | *1/*1 | *2/*2 | *1/*4 | *1/*3 | *1/*S12 | *1B/*1B | *1/*1 | |
| 11 | Astrolabe | | *2/*17 | *1/*1 | *1/*2 | | | | | |
| | PharmCAT | | | *2/*4B, *2/*17 | | | | rs41490561/rs4149056C | | |
| | Stargazer | *1/*5 | *1/*1 | *2/*17 | *1/*2 | *3/*3 | *9A/*S12 | *1/*17 | *1/*1 | *36, *60 |
| 12 | Astrolabe | | *1/*1 | *1/*2 | *2/*4 | | | | | |
| | PharmCAT | | | *1/*2, *1/*35 | | | | *5/*20, *5/*21 | | *36, *60 |
| | Stargazer | *1/*1 | *1/*2 | *1/*1 | *2/*4 | *3/*3 | *6/*S12 | *1/*15 | *1/*1 | |
| 13 | Astrolabe | | *1/*1 | *1/*2 | *1/*1 | | | | | |
| | PharmCAT | | *1/*2, *1/*35 | | | | | | | |
| | Stargazer | *1/*6 | *1/*2 | *1/*1 | *1/*122 | *3/*3 | *5/*9A | *1/*14 | *1/*1 | *1/*79 |
| 14 | Astrolabe | | *1/*1 | *1/*3 | *1/*1 | | | | | |
| | PharmCAT | | *1/*3, *1/*18 | | | | | rs4149056C/rs4149056C | | *36, *60 |
| | Stargazer | *2/*6 | *1/*3 | *1/*1 | *1/*1 | *3/*3 | *5/*S12 | *15/*15 | *1/*1 | |
| 15 | Astrolabe | | *1/*1 | *1/*1 | *1/*41 | | | | | |
| | PharmCAT | | | | | | | *1A/*18, *1A/*19 | | *36, *60, *60 |
| | Stargazer | *1/*5 | *1/*1 | *1/*1 | *1/*119 | *3/*3 | *S12/*S12 | *1/*1 | *1/*1 | |
| 16 | Astrolabe | | *1/*2 | *1/*2 | *1/*41 | | | | | |
| | PharmCAT | | *1/*2, *1/*35 | *1/*2 | | | | rs4149056C/rs4149056C | | *36, *60 |
| | Stargazer | *1/*9 | *1/*2 | *1/*2 | *1/*119 | *3/*3 | *9A/*9A | *15/*15 | *1/*1 | |
| 17 | Astrolabe | | *17/*17 | *1/*1 | *1/*2 | | | | | |
| | PharmCAT | | | *4B/*4B, | | | | rs4149056T/rs4149056C | | *60/*60 |
| | | | | *4B/*17, | | | | | | |
| | | | | *17/*17 | | | | | | |
| | Stargazer | *1/*6 | *1/*1 | *17/*17 | *1/*2 | *3/*3 | *9A/*S12 | *1/*17 | *1/*1 | |
| 18 | Astrolabe | | *1/*17 | *1/*1 | *1/*1 | | | | | |
| | PharmCAT | | | *1/*4B, *1/*17 | | | | *18/*18, *18/*19, *19/*19 | | *36, *60 |
| | Stargazer | *1/*1 | *1/*1 | *1/*17 | *1/*1 | *3/*3 | *9A/*S12 | *1/*1 | *1/*1 | |
| 19 | Astrolabe | | *1/*1 | *1/*2 | *1/*2 | | | | | |
| | PharmCAT | | *1/*2, *1/*35 | | | | | *1A/*18 | | *36, *60 |
| | Stargazer | *1/*1 | *1/*2 | *1/*1 | *1/*2 | *3/*3 | *9A/*9A | *1/*1B | *1/*1 | |
| 20 | Astrolabe | | *1/*17 | *1/*2 | *2/*2 | | | | | |
| | PharmCAT | | *1/*2, *1/*35 | *1/*4B, *1/*17 | | *3/*3 | | *1A/*18 | *1/*1 | *1/*1 |
| | Stargazer | *1/*1 | *1/*2 | *1/*17 | *2/*2 | | *9A/*9A | *1/*1B | | |

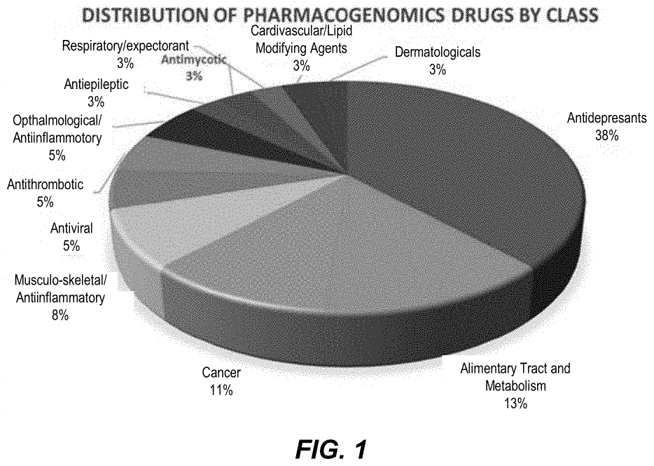

Based on the analysis of the nine pharmacogenes in Table 1, we identified 49 drugs from PharmGKB® PGx prescribing information for which CPIC® dosing guidelines were available. Twelve of these drugs did not have any prescription recommendation and we included the remaining 37 drugs with their pharmacogenomics-based dosage recommendations in our pipeline (see Table 3). More than one third of these drugs were categorized as antidepressants (38%), followed by alimentary tract and metabolism-related drugs (13%) and cancer drugs (11%). (FIG. 1)

TABLE 3
37 Drugs Selected for Inclusion in PharmaKU

| Drug | Class |
|---|---|
| amitriptyline | Nervous System/Psychoanaleptics/Antidepressants/Non- |
| | selective monoamine reuptake inhibitors |
| atazanavir | Antiinfectives For Systemic Use/Antivirals For Systemic |
| | Use/Direct Acting Antivirals/Protease inhibitors |
| atomoxetine | Nervous System/Psychoanaleptics/Psychostimulants. |
| | Agents Used For Adhd And Nootropics/Centrally acting |
| | sympathomimetics |
| capecitabine | Antineoplastic And Immunomodulating Agents/Antineo- |
| | plastic Agents/Antimetabolites/Pyrimidine analogues |
| celecoxib | Antineoplastic And Immunomodulating Agents/Antineo- |
| | plastic Agents/Other antineoplastic agent |
| citalopram | Nervous System/Psychoanaleptics/Antidepressants/ |
| | Selective serotonin reuptake inhibitors |
| clomipramine | Nervous System/Psychoanaleptics/Antidepressants/Non- |
| | selective monoamine reuptake inhibitors |
| clopidogrel | Blood And Blood Forming Organs/Antithrombotic |
| | Agents/Platelet aggregation inhibitors excl. heparin |
| codeine | Respiratory System/Cough And Cold Preparations/ |
| | Cough Suppressants. Excl. Combinations With Expecto- |
| | rants/Opium alkaloids and derivatives |
| desipramine | Nervous System/Psychoanaleptics/Antidepressants/Non- |
| | selective monoamine reuptake inhibitors |
| doxepin | Nervous System/Psychoanaleptics/Antidepressants/Non- |
| | selective monoamine reuptake inhibitors |
| efavirenz | Antiinfectives For Systemic Use/Antivirals For Systemic |
| | Use/Direct Acting Antivirals/Non-nucleoside reverse |
| | transcriptase inhibitors |
| escitalopram | Nervous System/Psychoanaleptics/Antidepressants/ |
| | Selective serotonin reuptake inhibitors |
| fluorouracil | Antineoplastic And Immunomodulating Agents/Antineo- |
| | plastic Agents/Antimetabolites/Pyrimidine analogues |
| flurbiprofen | Musculo-skeletal System/Antiinflammatory And Anti- |
| | rheumatic Products/Antiinflammatory And Antirheumatic |
| | Products. Non-steroids/Propionic acid derivatives |
| fluvoxamine | Nervous System/Psychoanaleptics/Antidepressants/ |
| | Selective serotonin reuptake inhibitors |
| fosphenytoin | Nervous System/Antiepileptics/Antiepileptics/Hydantoin |
| | derivatives |
| imipramine | Nervous System/Psychoanaleptics/Antidepressants/ |
| | Non-selective monoamine reuptake inhibitors |
| lansoprazole | Alimentary Tract And Metabolism/Drugs for Acid Related |
| | Disorders/Drugs for Peptic Ulcer And Gastro-oesophageal |
| | Reflux Disease (gord)/Proton pump inhibitors |
| lornoxicam | Musculo-skeletal System/Antiinflammatory And Anti- |
| | rheumatic Products/Antiinflammatory And Antirheumatic |
| | Products. Non-steroids/Oxicams |
| meloxicam | Musculo-skeletal System/Antiinflammatory And Anti- |
| | rheumatic Products/Antiinflammatory And Antirheumatic |
| | Products. Non-steroids/Oxicams |
| nortriptyline | Nervous System/Psychoanaleptics/Antidepressants/Non- |
| | selective monoamine reuptake inhibitors |
| omeprazole | Alimentary Tract And Metabolism/Drugs for Acid Related |
| | Disorders/Drugs For Peptic Ulcer And Gastro-oesophageal |
| | Reflux Disease (gord)/Proton pump inhibitors |
| ondansetron | Alimentary Tract And Metabolism/Antiemetics And Anti- |
| | nauseants/Antiemeties And Antinauseants/Serotonin |
| | (5HT3) antagonists |
| pantoprazole | Alimentary Tract And Metabolism/Drugs for Acid Related |
| | Disorders/Drugs for Peptic Ulcer And Gastro-oesophageal |
| | Reflux Disease (gord)/Proton pump inhibitors |
| paroxetine | Nervous System/Psychoanaleptics/Antidepressants/ |
| | Selective serotonin reuptake inhibitors |
| phenytoin | Nervous System/Antiepileptics/Antiepileptics/Hydantoin |
| | derivatives |
| piroxicam | Sensory Organs/Ophthalmologicals/Antiinflammatory |
| | Agents/Antiinflammatory agents, non-steroids |
| sertraline | Nervous System/Psychoanaleptics/Antidepressants/ |
| | Selective serotonin reuptake inhibitors |
| simvastatin | Cardiovascular System/Lipid Modifying Agents/Lipid |
| | Modifying Agents. Plain/HMG CoA reductase inhibitors |
| tacrolimus | Dermatologicals/Other Dermatological Preparations/ |
| | Agents for dermatitis, excluding corticosteroids |
| tamoxifen | Antineoplastic And Immunomodulating Agents/Endocrine |
| | Therapy/Hormone Antagonists And Related Agents/Anti- |
| | estrogens |
| tenoxicam | Sensory Organs/Ophthalmologicals Antiinflammatory |
| | Agents/Antiinflammatory agents, non-steroids |
| trimipramine | Nervous System/Psychoanaleptics/Antidepressants/ |
| | Non-selective monoamine reuptake inhibitors |
| tropisetron | Alimentary Tract And Metabolism/Antiemetics And |
| | Antinauseants/Antiemetics And Antinauseants/Serotonin |
| | (5HT3) antagonists |
| voriconazole | Antiinfectives For Systemic Use/Antimycotics For |
| | Systemic Use/Antimycotics For Systemic Use/Triazole |
| | derivatives |
| warfarin | Blood And Blood Forming Organs/Antithrombotic |
| | Agents/Antithrombotic Agents/Vitamin K antagonists |

### Example 2

### Using PharmaKU

Users can input the individual WGS VCF files through the web portal, which can be accessed securely from: http://ppgr.dasmaninstitute.org. Access can be provided upon request.

It is assumed that all VCF inputs meet minimum quality, requirements and have a coverage of at least 30×. Miles should be single sample VCF files in hg19 or GRCh38 reference format. The diplotypes called and the authenticity of the final report largely depend on the credibility of the input file.

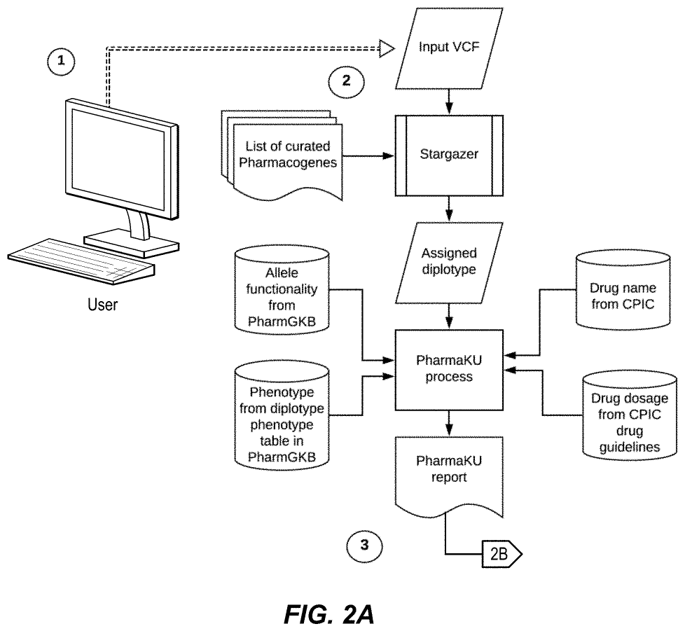

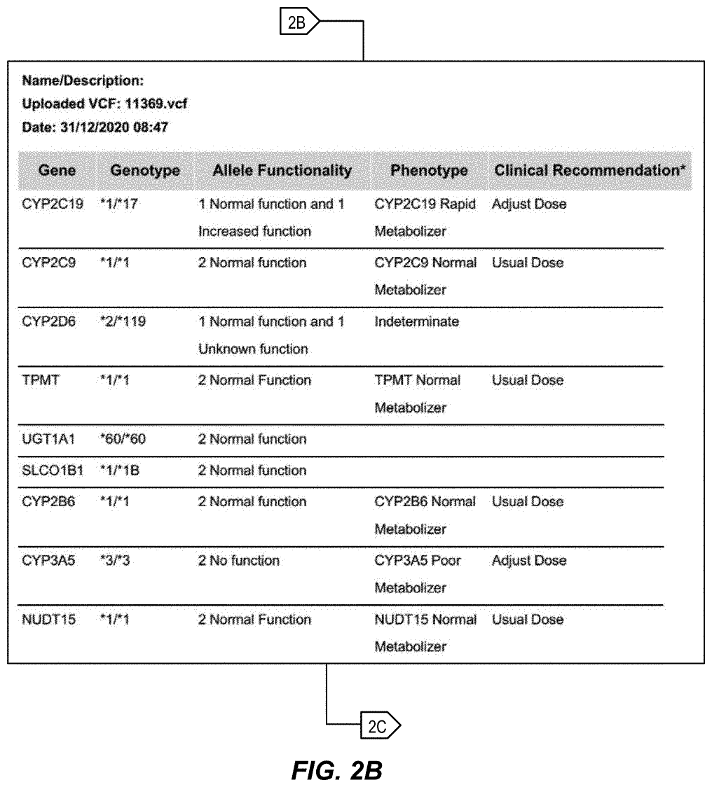

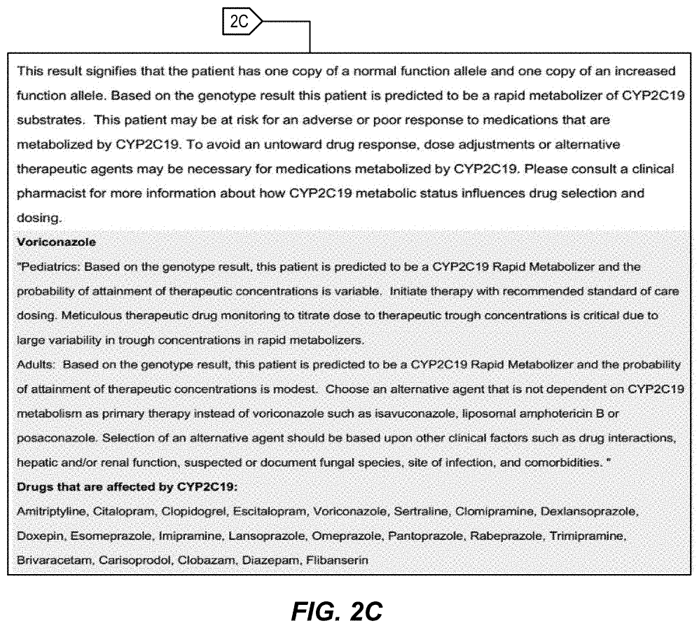

In the background, the software performs two tasks (FIG. 2). The main task involves the follow steps: inferring diplotypes for the nine pharmacogenes based on the input VCF file and then retrieving information corresponding to the called diplotype from PharmGKB® and the CPIC®. The process gathers phenotype and allele functionality information for the corresponding gene-diplotype pair from diplotype-phenotype tables obtained from PharmGKB®. From the list of 37 drugs that are affected by the nine pharmacogenes, and for which CPIC® drug dosage guidelines are available. The dosage recommendations are updated in the final report. These drugs have the FDA label “Actionable” or “Informative”, and are drugs for which PharmGKB® has made available pre- and post-test

The final report consists of two sections. The first section gives a summary of the genes with identified diplotype calls: allele functionality corresponding to the star alleles (unknown/uncertain/normal/no/increased/decreased function): phenotype status corresponding to allele functionality (indeterminate/poor/normal/intermediate/rapid/ultrarapid metabolizer) and clinical recommendations suggesting usual dose (for normal metabolizer) or adjust dose (for other phenotypes). There is an exception in the nomenclature of phenotype status in the SLCO1B1 gene according to the diplotype-phenotype file, where instead of the above, the conventions used are indeterminate/possibly decreased/decreased/normal/possibly increased/increased/possibly poor/poor function. The second section provides a detailed interpretation of the findings (consult note). For each gene listed in the first section, this will detail the consequence of the genotype on the allele functionality and phenotype. Wherever possible, dosage suggestions and changes are also recommended (see FIGS. 2B-2C).

### Example 3

### Analysis of Major Pharmacogenes Using 20 Reports

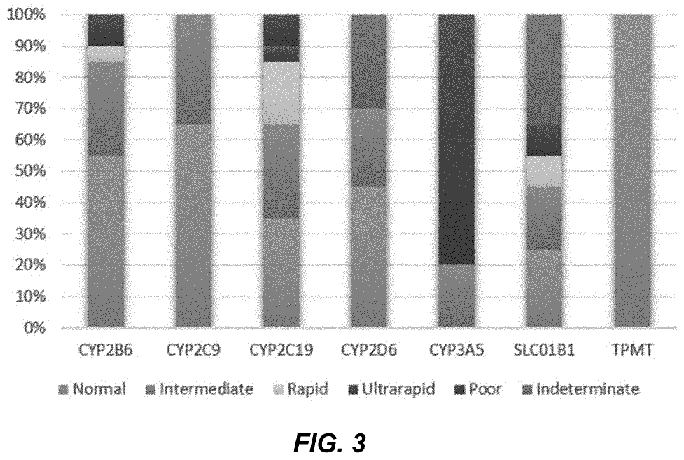

We compiled the reports generated from 20 WGS VCF files to determine the type of metabolizers and phenotype status for seven of the nine pharmacogenes found in these individuals (FIG. 3). DPYD and UGT1A1 were left out, as there was no phenotype status corresponding to the called diplotypes. It was observed that, in most of the genes, most of the samples exhibited normal metabolizer activity. However, for CYP3A5, almost 80% of the samples were poor metabolizers. CYP3A5 has known variants that modulate the activity of the drug tacrolimus, an antirejection medication for liver transplantation. One of the reported complications in interpreting CYP3A5 genotyping results is that most of the individuals involved in drug trials were of European descent and were therefore more likely to have the CYP3A5*3/*3 genotype, which predicts a poor metabolizer status. Hence, unlike other CYP enzymes, CYP3A5 variant and tacrolimus prescription pose an exception, wherein a CYP3A5 expresser (normal or intermediate metabolizer) would require a higher recommended starting dose and a CYP3A5 non-expresser (poor metabolizer) would require the standard recommended starting dose.

It is to be understood that the web-based pharmacogenomics tool is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A system for providing personalized medication dosing recommendations for a patient, comprising:
a server;
non-transitory memory configured to store executable instructions and pharmacogenomic data; and
a processor in communication with the server and the non-transitory memory;
wherein the server is adapted to receive information related to the patient from a user including a hole genome sequence file and communicate information to the user in the form of a personalized pharmacogenomics report;
wherein the executable instructions include instructions for the processor comprising:
receiving genomic information in either hg38 format or hg19 format from the user;
extracting diplotypes from the genomic information;
assigning allele functionality, phenotype, drug names, and recommended dosage information based upon the extracted diplotypes and a set of stored pharmacogenomic data;
generating a personalized pharmacogenomics report personalized for the patient comprising suggested dosing adjustments to at least one medication; and
delivering the personalized pharmacogenomics report to the user.

2. The system for providing personalized medication dosing recommendations as recited in claim 1, wherein the stored pharmacogenomic data comprises data relating to a selected set of genes.

3. The system for providing personalized medication dosing recommendations as recited in claim 2, wherein the selected set or genes is selected from the group consisting of CYP2B6, CYP2C19, CYP2C9, CYP2D6, CYP3A5, DPYD, SLCO1B1, TPMT, and UGT1A1, and a combination thereof.

4. The system or providing personalized medication dosing recommendations as recited in claim 2, wherein the stored pharmacogenomic data comprises data relating to CYP2B6, CYP2C19, CYP2C9, CYP2D6, CYP3A5, DPYD, SLCO1B1, TPMT, and UGT1A1.

5. The system for providing personalized medication dosing recommendations as recited in claim 1, wherein the suggested dosing adjustments include adjustments in dosing of at least one medication selected from the group consisting of amitriptyline, atazanavir, atomoxetine, capecitabine, celecoxib, citalopram, clomipramine, clopidogrel, codeine, desipramine, doxepin, efavirenz, escitalopram, fluorouracil, flurbiprofen, fluvoxamine, fosphenytoin, imipramine, lansoprazole, lornoxicam, meloxicam, nortriptyline, omeprazole, ondansetron, pantoprazole, paroxetine, phenytoin, piroxicam, sertraline, simvastatin, tacrolimus, tamoxifen, tenoxicam, trimipramine, tropisetron, voriconazole, and warfarin.

6. The system for providing personalized medication dosing recommendations as recited in claim 1, wherein the genomic information is received in hg38 format.

7. The system for providing personalized medication dosing recommendations as recited in claim 1, wherein the genomic information is received in hg19 format.

8. The system for providing personalized medication dosing recommendations as recited in claim 1, wherein the patient and the user are the same or are different.

9. The system for providing personalized medication dosing recommendations as recited in claim 8, wherein the patient and the user are different and are remote from one another.

10. A method for providing personalized medication dosing recommendations, comprising:
receiving genomic information in either hg18 format or hg19 format from a user;
extracting diplotypes from the genomic information;
assigning allele functionality, phenotype, drug names, and recommended dosage information based upon the extracted diplotypes and a set of stored pharmacogenomic data;
generating a personalized pharmacogenomics report personalized for the user comprising suggested dosing adjustments to at least one medication; and
delivering the personalized pharmacogenomics report to the user.

11. The method for providing personalized medication dosing recommendations as recited in claim 10, wherein the stored pharmacogenomic data comprises data relating to a selected set of genes.

12. The method for providing personalized medication dosing recommendations as recited in claim 11, wherein the selected set of genes is selected front the group consisting of CYP2B6, CYP2C19, CYP2C9, CYP2D6, CYP3A5, DPYD, SLCO1B1, TPMT, and UGT1A1, and a combination thereof.

13. The method for providing, personalized medication dosing recommendations as recited in claim 11, wherein the stored pharmacogenomic data comprises data relating to CYP2B6, CYP2C19, CYP2C9, CYP2D6, CYP3A5, DPYD, SLCO1B1, TPMT, and UGT1A1.

14. The method for providing personalized medication dosing recommendations as recited in claim 10, wherein the suggested dosing adjustments include adjustments in dosing of at least one medication selected from the group consisting of amitriptyline, atazanavir, atomoxetine, capecitabine, celecoxib, citalopram, clomipramine, clopidogrel, codeine, desipramine, doxepin, efavirenz, escitalopram, fluorouracil, flurbiprofen, fluvoxamine, fosphenytoin, imipramine, lansoprazole, lornoxicam, meloxicam, nortriptyline, omeprazole, ondansetron, pantoprazole, paroxetine, phenytoin, piroxicam, sertraline, simvastatin, tacrolimus, tamoxifen, tenoxicam, trimipramine, tropisetron, voriconazole, and warfarin.

15. The method for providing personalized medication dosing recommendations as recited in claim 10, wherein the genomic information is received in hg38 format.

16. The method for providing personalized medication dosing recommendations as recited in claim 10, wherein the genomic information is received in hg19 format.

17. The method for providing personalized medication dosing recommendations as recited in claim 10, wherein the patient and the user are the same or are different.

18. The method for providing personalized medication dosing recommendations as recited in claim 17, wherein the patient and the user are different and are remote from one another.

19. A non-transitory memory configured to store executable instructions, the executable instructions programming a processor to perform a method comprising:
receiving genomic information in either hg38 format or hg19 format from a user;
extracting diplotypes from the genomic information;
assigning allele functionality, phenotype, drug names, and recommended dosage information based upon the extracted diplotypes and a set of stored pharmacogenomic data;
generating a personalized pharmacogenomics report personalized for the user comprising suggested dosing adjustments to at least one medication; and
delivering the personalized pharmacoeconomics report to the user.

20. The non-transitory memory configured to store executable instructions as recited in claim 19, wherein the stored pharmacogenomic data comprises data relating to CYP2B6, CYP2C19, CYP2C9, CYP2D6, CYP3A5, DPYD, SLCO1B1, TPMT, and UGT1A1.
